(19) United States
(12) Patent Application Publication
(10) Pub. No.: US 2004/0132068 A1
(43) Pub. Date: Jul. 8, 2004

(54) Method and device for preparing biological samples for analysis

(76) Inventor: Walter Schubert, Biederitz (DE)

Correspondence Address: WOMBLE CARLYLE SANDRIDGE & RICE, P.O. Box 7037, Atlanta, GA, 30357-0037, US

(21) Appl. No.: 10/686,865

(22) Filed: Oct. 16, 2003

Related U.S. Application Data
(63) Continuation of application No. PCT/EP03/00357, filed on Jan. 15, 2003.

(30) Foreign Application Priority Data
Jan. 22, 2002 (EP) 02001519.4

(51) Int. Cl. C12Q 1/68; G01N 1/30; G01N 33/48; C12N 1/08

(52) U.S. Cl. 435/6; 435/270; 435/40.5

(57) ABSTRACT

The invention relates to a method for preparing biological samples for analysis, comprising the following steps: (a) the biological sample is placed on a two-dimensional support; (b) application of a protein-precipitating or denaturing first solution L1 to the biological sample at a first temperature T1 for a predetermined first time period Z1; (c) the protein-precipitating or denaturing first solution L1 is then left, or more solution is applied to the biological sample or a protein-precipitating or denaturing second solution L2 is applied to the biological sample at a temperature T2, for a predetermined second time period Z2, whereby T2 is lower than T1 and Z2 is longer, equal to or shorter than Z1; and (d) drying of the sample. The invention also relates to a device for carrying out a method for the preparation of biological samples for analysis, in accordance with one of the aforementioned claims, said device (10) comprising at least one chamber (12) for receiving the biological sample or samples that has/have been applied to at least one support and comprising at least one temperature controller (14) for controlling and adjusting the temperature inside the chamber (12).

### CROSS-REFERENCE TO RELATED APPLICATIONS

This application is a continuation of PCT application no. PCT/EP03/00357 (designating the U.S.), filed on Jan. 15, 2003; which application claims priority on European application no. 02001519.4, filed on Jan. 22, 2002.

### INCORPORATION BY REFERENCE

The specification of PCT/EP03/00357 as well as the specification of European patent application no. 02001519.4 are incorporated herein in their entirety, by this reference.

### FIELD OF THE INVENTION

The invention relates to a method for preparing biological samples for analysis and a device for performing a method for preparing biological samples for analysis.

### BACKGROUND OF THE INVENTION

Since the complete sequencing of the human genome and that of other species, the qualitative and quantitative analysis of proteins in a given cell or tissue sample is increasingly gaining importance in research and industry.

With regard to the protein-chemical analysis methods, modem, so-called large-scale proteomics methods are prominent. These methods are based on a breakdown of a given cell or tissue sample, an extraction and denaturing of the proteins with the aid of different reagents and the separation of the individual proteins with the aid of, for example, two-dimensional gel electrophoresis methods. Each individual protein that exists in the original protein mixture in a sufficient quantity and size and with an adequate migration capability, is shown to exist within these gels as a blot at a characteristic location with regard to the size and the net charge. In the practical work of what is known as proteome research, up to several thousand proteins can be separated from a sample in this way. Based on a comparison of different states of a tissue, it is theoretically possible to determine differences in the protein expression by way of a superposition, which is accurate as to the location, of the individual gels with the aid of an appropriate software. In particular, it should be possible in this process to identify newly expressed proteins and the no longer expressed or over-expressed proteins by way of a comparison of locations.

In every-day practical analytical work, however, there are significant limitations with regard to this objective. For example, the known analysis methods exhibit limited reproducibilities because, owing to the lack of automation, there are significant inaccuracies in the individual analysis steps. With regard to the position of individual proteins, for example, deviations of up to several per cent result. With regard to the relative quantity of the proteins, deviations of up to twenty per cent result.

Apart from the difficulty in performing the known methods technically, it is also their dynamism that is insufficient, i.e. very rare proteins cannot be shown side-by-side with frequently occurring proteins. These limitations make it impossible to compare in an automated way a larger number of e.g. gels with the aid of a suitable software that relies on reproducible positions of individual proteins. The positions of individual protein blots, which deviate slightly form each other, increase the uncertainly of comparative analyses. In proteome research, however, one relies on the comparison of very large quantities of cell tissue, so the aforementioned limitations of the known methods are not tolerable.

One essential reason for the variability or the inaccuracies and limited reproducibilities of the known analysis methods is in particular also the non-standardised sample preparation.

### SUMMARY OF THE INVENTION

It is therefore an object of the present invention to further develop and make available a method for preparing biological samples for analysis, which ensures homogeneous and standardised and, where applicable, fully automated sample preparation.

Furthermore, it is an object of the present invention to provide a corresponding device for performing the new method for preparing biological samples for analysis.

This object is achieved by a method having the features of claim 1 and a device having the features of claim 11.

Advantageous embodiments are described in the subclaims.

In a method according to the present invention for preparing biological samples for analysis, the following steps are performed: a) placing the biological sample on a two-dimensional support; b) applying a protein-precipitating or denaturing first solution L1 to the biological sample at a first temperature T1 for a predetermined first time period Z1; c) leaving the protein-precipitating or denaturing solution L1 or applying more protein-precipitating or denaturing solution L1, or applying a second protein-precipitating or denaturing solution L2 to the biological sample at a second temperature T2 for a predetermined second time period Z2, with T2 being lower than T1 and Z2 being longer, equal to or shorter than Z1; and d) drying the sample. Through the method according to the present invention for preparing samples it is ensured that a homogeneous, highly standardised sample preparation is performed and that correspondingly homogeneous samples are being created. As a consequence it is possible that by way of corresponding computer programs the proteins to be analysed and examined can be identified and qualified in a significantly better way. Measurements have shown that the error likelihood of protein recognition decreases by a factor of 3 to 4. Moreover, the number of automatically identified proteins increases by at least 16%. Finally, the new method for preparing samples enables a full automation of sampling and hence the possibility to compare quantitative results between different laboratories.

In an advantageous embodiment of the method according to the present invention, a drying of the sample takes place between the process steps a) and b) as process step a1) and/or between the process steps b) and a) as process step b1). It has shown that, as a result, a further homogenisation and also concentration of the biological sample to be examined takes place, whereby the drying in accordance with process steps a1), b1) or d) can take place by way of air or vacuum drying.

In a further advantageous embodiment of the method according to the present invention, the sample is frozen as process step b2) after process steps a) or a1). This way, too, a homogeneous protein concentration and a more stable protein precipitation can advantageously be achieved, it being possible for the biological sample to be a cell or tissue sample or a mixture of proteins or nucleic acids or a mixture of macromolecules consisting of proteins and/or carbohydrates and/or fats and/or nucleic acids.

In a further advantageous embodiment of the method according to the present invention the solutions L1 and/or L2 are organic solvents and/or solutions with critical pH values and/or solutions with critical ion concentrations and/or salt solutions and/or solutions containing metal ions. For the performance of the method according to the present invention for preparing samples, the organic solvents methanol, ethanol, butanol and acetone have proven especially advantageous. It is the dissolved salts of picric acid, gallotannic acid, tungstic acid, molybdenum acid, trichloroacedic acid, perchloric acid and sulphosalicylic acid that are particularly employed as salt solutions. A range of −10° C. to 60° C. has proven an advantageous temperature range T1.

In a further advantageous embodiment of the method according to the present invention the biological samples, after process step d), are subjected to a protein and/or nucleic acid determination method and/or a protein-chemical separation method and/or a method for the in-situ analysis of cell structures.

A device according to the present invention for performing the described method for preparing biological samples for analysis exhibits at least one chamber for receiving the sample or samples applied to at least one support and at least one temperature controller for controlling and adjusting the temperature inside the chamber. This ensures in an advantageous way that the biological sample to be treated is exposed to different temperature ranges inside the chamber.

In an advantageous embodiment of the device according to the present invention, there are arranged several chambers in series and behind one another. It is, however, also possible to have several chambers arranged above one another, or to have at least one separation wall arranged within an individual chamber. As a result of the formation of several chambers it is advantageously possible to assign each chamber a corresponding temperature range or another reaction range. This increases sample throughput because it is not necessary, for example, to cool down or heat up an individual chamber in order to achieve different temperature ranges.

In a further advantageous embodiment of the device according to the present invention there are arranged several supports on one or several sample slides. This, too, leads to a significant increase in sample throughput, it being possible for the individual process steps to be executed and controlled manually, semi-automatically or automatically by the device according to the present invention.

### BRIEF DESCRIPTION OF THE DRAWINGS

Further details, features and advantages of the invention follow from the embodiments depicted and described in the following figures, which show in:

FIG. 1 a schematic representation of a device according to the present invention for performing a method for preparing biological samples for analysis according to a first embodiment;

FIG. 2 a schematic representation of the device according to FIG. 1 and a sample slide transferred into a first chamber, and

FIG. 3 a schematic representation of the device according to FIG. 1 with the sample slide inside a second chamber;

FIG. 4 a schematic representation of a device according to the present invention for performing a method for preparing biological samples for analysis according to a second embodiment; and

FIG. 5 a schematic representation of the operating principle of the device according to FIG. 4.

### DETAILED DESCRIPTION OF SOME EMBODIMENTS

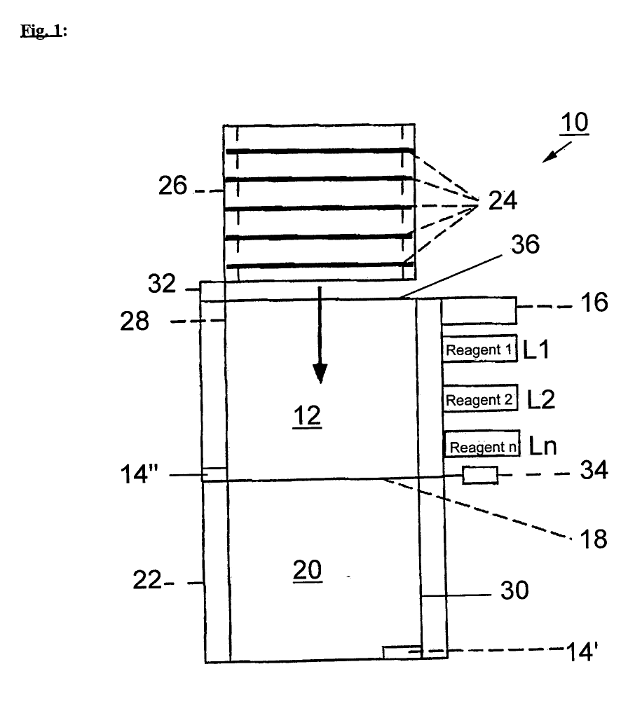

FIG. 1 shows in a schematic representation a device 10 for performing a method for preparing biological samples for analysis with a first chamber 12 and a second chamber 20 located inside a housing 22. A sample slide 26 with a plurality of supports 24, to which biological samples have been applied, may be moved into and out of the first chamber 12 and the second chamber 20 via the rails 28, 30, the movement of the sample slide 26 being accomplished by the first motor 32.

It can also be seen that the housing 22 or the first chamber 12 can be closed by means of a lid 36. The first chamber 12 is furthermore equipped with a vacuum pump 16 and several connections for introducing different protein-precipitating or denaturing solutions L1, L2, Ln, the connections being designed such that the solutions can be introduced into and removed from the chambers 12, 20. It can also be seen that the first chamber 12 is separated from the second chamber 20 by means of a movable separation wall 18. The movement of the separation wall 18 is controlled via a second motor 34. In the area of the second chamber 20 a temperature controller 14′ is arranged for controlling the temperature inside the chambers 12, 20. Optionally, a corresponding temperature controller 14″ can also be arranged inside the first chamber 12, it being possible for the temperatures inside the chambers 12, 20 to be set within a temperature range of −10° C. to 60° C. Apart form the aforementioned automatic or motorised removal of the separation wall 18, this can, of course, also be performed manually.

It is, however, also possible that instead of the two chambers 12, 20 only a single chamber is envisaged (not shown).

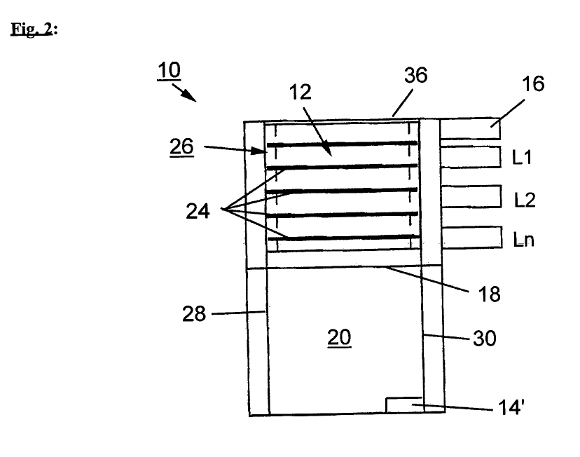

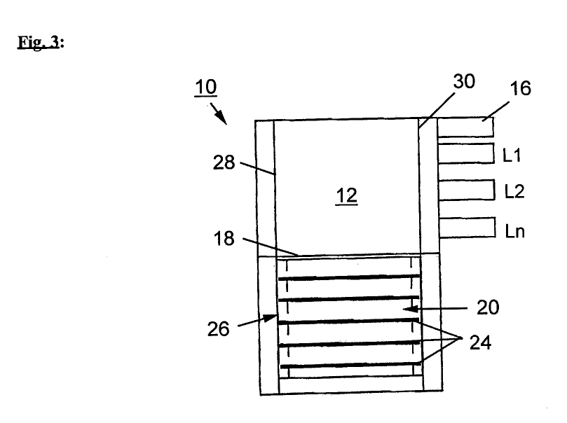

In the following the operating principle of the first embodiment described here will be explained in more detail by way of FIGS. 1 to 3.

In the case of fully automatic control of the device 10, the first motor 32 moves the slide box 26 in programmed time periods Z1, Z2 from the first chamber 12 into the second chamber 20 and from the second chamber 20 back into the first chamber 12, it being possible for this procedure to be repeated any number of times. By activating the vacuum pump 16 a vacuum can be generated either in chamber 12 or in both chambers 12, 20, depending on the position of the separation wall 18, the generation of the vacuum serving either to dry the samples on the supports 24 or in the sample slide 26. The same applies mutatis mutandis to the supply and removal, by suction, of the solutions L1, L2, Ln. Here, too, depending on the position of the separation wall 18, either only the first chamber 12 or both chambers 12, 20 can be filled with the corresponding solutions L1, L2, Ln and be emptied.

In a first process step a) the biological samples are applied to a two-dimensional support 24. The biological samples are usually a cell or tissue sample or a mixture of proteins or nucleic acids or a mixture of macromolecules consisting of proteins and/or carbohydrates and/or fats and/or nucleic acids. Cells from a cell culture can, for example, be received in a buffer, the cell density being set at e.g. 3×10⁸ cells. It is, however, also possible for a cryostat tissue section to be used as a sample. The choice of the number of cells or the number of tissue sections depends on the objective, i.e. to which other method the biological samples are supplied after the completion of sample preparation. This can generally be a protein- and/or nucleic acid determination method and/or a protein-chemical separation method and/or a method for the in-situ analysis of cell structures. The cells dissolved in the buffer are applied evenly to the support 24 with the help of a pipette. Alternatively, one or several tissue sections are received on the support 24. Depending on the size of the sample slide 26, it is thus possible to receive a plurality of supports 24. Prior to the transfer of the sample slide 26 into the device 10, the separation wall 18 is shifted outward by pulling, so that the second chamber 20 becomes accessible from the first chamber 12 arranged above it. The second chamber 20 is then filled with the first protein-precipitating or denaturing solution L1 to just below the top chamber rim. With the embodiment described here, the first solution L1 consists of an organic solvent, e.g. methanol, ethanol, butanol or acetone. However, it is also possible for the solutions L1 and L2 to not only consist of organic solvents but also of solutions with critical pH values and/or solutions with critical ion concentrations and/or salt solutions and/or metal ion-containing solutions, it being possible for the salt solutions to contain dissolved salts of picric acid, gallotannic acid, tungstic acid, molybdenum acid, trichloroacedic acid, perchloric acid or sulphosalicylic acid.

In a next working step the separation wall 18 is pushed back into its starting position, i.e. into a final position to separate the first chamber 12 from the second chamber 20. Next, the slide box 26 is placed into the rails 28, 30 of the device 10 and transferred into the first chamber 12. Also, the lid 36 of the device 10 is closed. Next, in a further working step, a vacuum is generated inside the first chamber 12 with the aid of the vacuum pump 16. This way the biological samples are dried for a first time in accordance with a process step a1). Following the completion of this drying process a1) the vacuum is removed from the first chamber 12. Finally, the separation wall 18 between the first chamber 12 and the second chamber 20 is again removed and the slide box 26 is lowered into the second chamber 20 which is filled with the first solution L1. This way the protein-precipitating or denaturing first solution L1 is applied to the biological samples and the supports 24 at a first temperature T1, which, in the present case, is the room temperature. Through the contact with the organic solvent L1 the proteins of the samples have water extracted from the hydratation jacket. After a predetermined time period Z1, which can for example be 10 seconds, the slide 26 is again pulled into the chamber 12. This completes process step b). Owing to the very short exposure time there is only a partial, gentle water extraction, so that the three-dimensional structure of the proteins in the cells is not influenced or only slightly influenced. This way the proteins become homogeneously accessible for the subsequent process step c). Before said process step c) is performed, the separation wall 18 is again pushed back in, so that the two chambers 10, 20 are again separated. According to a process step b1), the sample is now once again dried with the aid of the vacuum pump 16. Finally, following the completion of the drying process and the removal of the vacuum from the first chamber 12, the separation wall 18 between the chambers 10, 20 is again removed.

In the following working step the temperature inside the chambers 10, 20 is lowered to and set at −20° C. using the temperature controller 14′. As the biological samples are arranged in the gaseous phase of the organic solvent L1 in the first chamber 12, they are frozen in accordance with a process step b2). It is also possible to freeze the samples by supplying liquid nitrogen. The same applies mutatis mutandis to the supply of liquid isopentane at approx. −130° C. Later on, these liquids have to be removed once again from the system. In the second chamber 20, owing to the usually much lower freezing point, the organic solvent L1 is in its liquid state. This is true in particular where acetone is used as an organic solvent. Thereafter, the slide box 26 with the biological samples is lowered into the second chamber 20, so that in accordance with process step c) the protein-precipitating or denaturing first solution L1 is applied further to the biological sample at the second temperature T2. Now, the samples remain in the second chamber 20 for a predetermined time period Z2, which, for the embodiment described, can be approx. 10 minutes. Owing to the additional application of the first solution L1 at a low temperature T2, the water jacket of the cellular proteins is extracted in situ in a gentle way, which, because of the prepared process step b) is homogeneous and complete, the three-dimensional structure of proteins and protein complexes of the treated biological sample being largely retained.

Thereafter, the slide box 26 is again transferred into the first chamber 12, the separation wall again being pushed between the chambers 12, 20 and locked in place. In a final process step d) the ready-prepared samples are now dried. This is again done with the aid of the vacuum pump 16. However, it is also possible for the drying of the samples to take place by air drying.

Yet the embodiment of the device 10 also allows not only one solution L1 to be used but a plurality of different solutions L2 to Ln to be sequentially or simultaneously filled in and removed by suction.

However, it is also possible to dispense with the separation wall 18 for the device 10. In this case the device 10 consists only of one chamber 12 (not shown) and a temperature controller 14′, the upper half of the chamber 12 being envisaged for the gaseous phase of the solutions L1, L2 to Ln to be filled in and removed by suction, and the bottom half being envisaged for the liquid phase of said solutions. The entire process is controlled manually in predetermined time periods. For this version of the device 10 it is also possible to dispense with the vacuum pump and the motors 32, 34.

Furthermore, by varying the height of the chambers 12, 20 of the device 10 shown in the FIGS. 1 to 3, it is possible to use different slide boxes 26 with a variable carrying capacity with regard to the number of supports 24. This increases the number of samples that can be processed in parallel. This way, it is possible, for example, to have between ten and fifty samples per device 10 processed simultaneously. It is also possible to increase the number of the samples to be processed to any number by operating the device 10 in parallel. For this, an individual vacuum pump 16 can be linked to a plurality of devices 10 using corresponding connections.

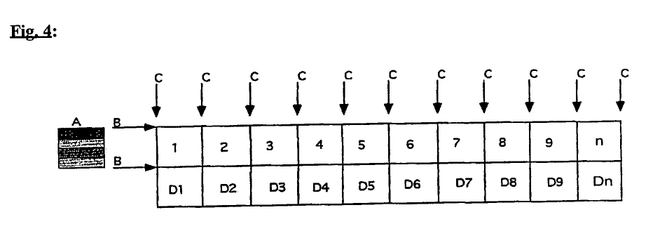

FIG. 4 shows in a schematic representation a device for performing a method for preparing biological samples for analysis in accordance with a second embodiment. One can see that several chambers 1, 2, 3 . . . , n are arranged in series and behind each other, it being possible for the individual chambers 1, 2, 3 . . . , n to be closed with corresponding lids D1, D2, D3 . . . , Dn.

In the following, the second embodiment shown here will be described in more detail with regard to its operating principle. A slide box A with the samples contained therein or the sample-containing supports is inserted into the rails B and positioned above the chamber 1. Thereafter, the slide box A is lowered into the chamber 1 and the chamber lid D1 is pushed over the chamber 1 by means of the guide rail C. Next, a vacuum is created inside the chamber 1. Following the completion of the vacuum drying process and the air flooding of the chamber 1, the lid D1 is moved back into its starting position. The slide box A can then be lifted out of the chamber 1 back into the guide rail B. When the slide box A has been moved to above the chamber 2 it is lowered into the chamber 2. A lid D2 again closes the chamber 2. Inside the chamber 2 the protein-precipitating or denaturing first solution L1 is applied according to process step b). After removal of the sample slide A from the chamber 2 and a corresponding lowering of the sample slide A into the chamber 3, the samples are dried in the chamber 3 in accordance with process step b1). Chamber 4 serves to perform the process step c), i.e. another application of a protein-precipitating or denaturing solution at a second temperature T2, which is lower than the first temperature T1, which, in the present case, has prevailed inside the chamber 2. In the device shown, the chamber 5 serves as a vacuum chamber for further sample drying in accordance with process step d). All other chambers can be used for further sample treatment. In chamber 6, for example, a buffer or a second solution L2 can be applied to the samples. The same applies mutatis mutandis to chambers 7 to 9 with further buffer solutions or further protein-precipitating or denaturing solutions. Moreover, there may be chambers designed for the asservation of the samples after a cycle. Moreover, chambers can contain a so-called “cell/tissue sampler” which receives the treated samples from the support in a test glass or a centrifuge tube.

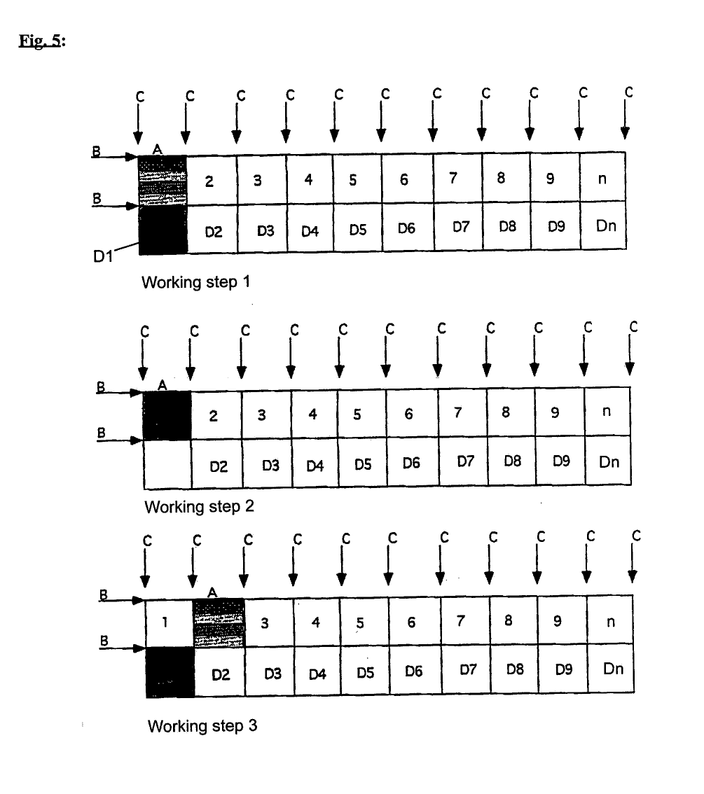

One can see that the same working steps as in the device 10 shown in FIGS. 1 to 3 can be performed by the linear device according to the second embodiment. However, the individual reactions are carried out in separate chambers 1, 2, 3 . . . , n specific for each reaction (cf. FIG. 5).

In another embodiment not shown the individual chambers of the device can also be arranged in a circle within a so-called carousel.

Although shown in some embodiments in the figures, it will be recognized from the description set out herein that the present invention contemplates methods for preparing biological samples for analysis and corresponding devices which, although not constructed exactly as shown in the figures, function in substantially similar fashion to achieve substantially similar results as the methods and devices shown. All such changes are intended to fall within the spirit and scope of the following claims.

What is claimed is:

1. Method for preparing biological samples for analysis, comprising the following steps:
a) placing the biological sample on a two-dimensional support;
b) applying protein-precipitating or denaturing first solution L1 to the biological sample at a first temperature T1 for a predetermined first time period Z1;
c) leaving the protein-precipitating or denaturing solution L1 or applying more protein-precipitating or denaturing solution L1, or applying a protein-precipitating or denaturing solution L2 to the biological sample at a second temperature T2 for a predetermined second time period Z2, with T2 being lower than T1 and Z2 being longer, equal to or shorter than Z1; and
d) drying the sample.

2. Method according to claim 1, wherein a drying of the sample takes place between the process steps a) and b) as process step a1) and/or between the process steps b) and c) as process step b1).

3. Method according to claim 2, wherein said drying of the sample takes place by means of air or vacuum drying.

4. Method according to claim 1, wherein after said process steps b) or b1) as process step b2), the sample is frozen.

5. Method according to claim 1, wherein said biological sample is a cell or tissue sample or a mixture of proteins or nucleic acids or a mixture of macromolecules comprising proteins and/or carbohydrates and/or fats and/or nucleic acids.

6. Method according to claim 1, wherein said solutions L1 and/or L2 are organic solvents and/or solutions with critical pH values and/or solutions with critical ion concentrations and/or salt solutions and/or solutions containing metal ions.

7. Method according to claim 6, wherein said organic solvents are methanol and/or ethanol and/or butanol and/or acetone.

8. Method according to claim 6, wherein said salt solutions contain dissolved salts of picric acid and/or gallotannic acid and/or tungstic acid and/or molybdenum acid and/or trichloroacedic acid and/or perchloric acid and/or sulphosalicylic acid.

9. Method according to claim 1, wherein TI covers a temperature range of −10° C. to 60° C.

10. Method according to claim 1, wherein after said process step d), said biological samples are subjected to a protein and/or nucleic acid determination method and/or a protein-chemical separation method and/or a method for the in-situ analysis of cell structures.

11. Device for performing a method for preparing biological samples for analysis according to claim 1, wherein said device exhibits at least one chamber to receive the biological sample or samples applied to a support and at least one temperature controller for controlling and adjusting the temperature inside said chamber.

12. Device according to claim 11, wherein said chamber can be closed with a lid.

13. Device according to claim 11, wherein said device exhibits at least one vacuum pump to generate a vacuum inside said chamber.

14. Device according to claim 12, wherein said device exhibits at least one vacuum pump to generate a vacuum inside said chamber.

15. Device according to claim 11, wherein there is arranged inside said chamber at least one separation wall.

16. Device according to claim 15, wherein said separation wall can be removed or shifted manually or automatically.

17. Device according to claim 11, wherein several chambers (1, 2, 3 . . . , n) are arranged in series and behind each other.

18. Device according to claim 11, wherein several of said chambers are arranged above one another.

19. Device according to claim 11, wherein several of said supports are arranged on one or several sample slides.

20. Device according to claim 11, wherein the individual process steps are executed and controlled manually, semi-automatically or automatically by said device.
